(19) United States
(12) Patent Application Publication
(10) Pub. No.: US 2024/0180473 A1
(43) Pub. Date: Jun. 6, 2024

(54) APPARATUS, METHOD, AND COMPUTER READABLE RECORDING MEDIUM FOR MEASURING ECG-AXIS DEVIATION USING HILBERT TRANSFORM

(71) Applicants: Man-Rim CHOI, Busan (KR); Shin-Rim CHOI, Seoul (KR)

(72) Inventor: Man-Rim CHOI, Busan (KR)

(21) Appl. No.: 18/285,259

(22) PCT Filed: Jun. 2, 2021
(86) PCT No.: PCT/KR2021/006875

§ 371 Date: Oct. 1, 2023

(30) Foreign Application Priority Data
Mar. 30, 2021 (KR) 10-2021-0041177

(51) Int. Cl. A61B 5/352; A61B 5/353; A61B 5/355

(52) U.S. Cl. CPC A61B 5/352; A61B 5/353; A61B 5/355

(57) ABSTRACT

An apparatus for measuring the magnitude of an electrocardiogram signal using a Hilbert transform, according to an embodiment of the present invention, may comprise: a reception unit which receives a measured electrocardiogram signal; a transform unit which performs a Hilbert transform of the received electrocardiogram signal; and a control unit which obtains an electrocardiography (ECG)-axis deviation on the basis of the Hilbert-transformed electrocardiogram signal.

### FIELD OF INVENTION

The present invention relates to an apparatus and method for measuring ECG axial deviation using Hilbert transform, and a computer-readable recording medium.

### BACKGROUND OF INVENTION

In diagnosis using medical images (ultrasound, MT, CT), electrocardiography (ECG) signals are used to extract not only image information but also images at specific points in the body.

A waveform of the ECG signal may be displayed as a curve centered on a base line (BL) between a current and a potential difference generated by contraction of the heart. In general, P wave, Q wave, R wave, S wave and T wave are sequentially generated within one cycle of the ECG signal. P wave represents the contraction of the atrium, a series of Q wave, R wave and S wave (QRS complex) represent the contraction of the ventricles, while T wave represents characteristics appearing at the relaxation of the ventricles.

On the other hand, ECG axial deviation (Axis Deviation) is a symptom in which the electrical axis of the heart is tilted to the right or left than normal, and such ECG axial deviation is very important in diagnosing various diseases such as right ventricular hypertrophy, acute right ventricular strain, chronic obstructive lung disease (COPD), hyperkalemia, Wolf-Parkinson-White syndrome, dextrocardia, or the like, therefore, it is necessary to measure ECG axial deviation.

### SUMMARY OF INVENTION

### Technical Problem to be Solved

According to one embodiment of the present invention, there are provided ECG axial deviation measuring apparatus and method, which can obtain ECG axial deviation using Hilbert transform, and a computer-readable recording medium.

### Technical Solution

According to a first embodiment of the present invention, a reception unit for receiving the measured electrocardiography (ECG) signal; a converter for Hilbert-transforming the received ECG signal; and a control unit for obtaining an ECG axial deviation based on the Hilbert-transformed ECG signal.

According to an embodiment of the present invention, the control unit may include: a Nyquist diagram creation module for creating a Nyquist diagram in which the ECG signal value is a real value, while the Hilbert-transformed value of the ECG signal is an imaginary value; and a control module that calculates the ECG axial deviation based on the created Nyquist diagram.

According to an embodiment of the present invention, the control unit may further include an envelope creation module configured to create an envelope for the ECG signal and the Hilbert-transformed ECG signal.

According to an embodiment of the present invention, the control module may obtain the angle of a straight line that connects a point corresponding to a peak value of the envelope in the Nyquist diagram with an origin of the Nyquist diagram, as the ECG axial deviation.

According to an embodiment of the present invention, the control module may obtain an angle defined by a perpendicular line from a common tangent of two points of the Nyquist diagram to the origin of the Nyquist diagram, as the ECG axial deviation.

According to an embodiment of the present invention, when there are two or more common tangents, the control module may calculate the angle defined by the perpendicular line from an average tangent of two or more common tangents to the origin of the Nyquist diagram, as the ECG axial deviation.

According to an embodiment of the present invention, the control module may calculate an angle of a vector obtained by adding the sum of vectors up to each point corresponding to each peak value of Q wave, R wave and S wave of the ECG signal in the Nyquist diagram, and the sum of vectors up to each point corresponding to each peak value of Qi wave, Ri wave and Si wave of the Hilbert-transformed ECG signal in the Nyquist diagram, as the ECG axial deviation.

According to an embodiment of the present invention, the control module may determine the angle of a straight line that connects a point corresponding to the peak value of T wave of the envelope in the Nyquist diagram with the origin of the Nyquist diagram, as the ECG T wave axial deviation.

According to an embodiment of the present invention, the control module may determine the angle of a straight line that connects a point corresponding to the peak value of P wave of the envelope in the Nyquist diagram with the origin of the Nyquist diagram, as the ECG P wave axial deviation.

According to a second embodiment of the present invention, there is provided a method for measuring ECG axial deviation using Hilbert-transform, which includes: a first step of receiving the measured ECG signal in a reception unit; a second step of Hilbert-transforming the received ECG signal in a converter; a third step of calculating ECG axial deviation based on the Hilbert-transformed ECG signal in a control unit.

According to a third embodiment of the present invention, there is provided a computer-readable recording medium, in which a program for executing the above-described method for measuring ECG axial deviation using Hilbert transform is recorded.

### Effect of Invention

According to one embodiment of the present invention, the received ECG signal may be Hilbert-transformed, and electrocardiography (ECG) axial deviation may be obtained based on the Hilbert-transformed ECG signal.

### BRIEF DESCRIPTION OF DRAWINGS

FIG. 1 is a block diagram of an ECG axial deviation measuring apparatus using Hilbert Transform according to an embodiment of the present invention.

FIG. 2a is a diagram illustrating an exemplary ECG signal according to an embodiment of the present invention.

FIG. 2b is a diagram illustrating an exemplary Hilbert-transformed ECG signal and an envelope according to an embodiment of the present invention.

FIG. 2c is a diagram illustrating an exemplary Nyquist diagram according to an embodiment of the present invention.

FIGS. 3 to 7c are diagrams for explaining a process of obtaining the ECG axial deviation according to an embodiment of the present invention.

FIG. 8 is a flowchart illustrating a method for measuring ECG axial deviation using a Hilbert Transform according to an embodiment of the present invention.

### BEST MODE FOR CARRYING OUT INVENTION

Embodiments of the present invention may be modified in various other forms, and the scope of the present invention is not limited only to the embodiments described below. The shapes and sizes of elements in the drawings may be exaggerated for clearer description, and the elements indicated by the same reference numerals in the drawings are the same elements.

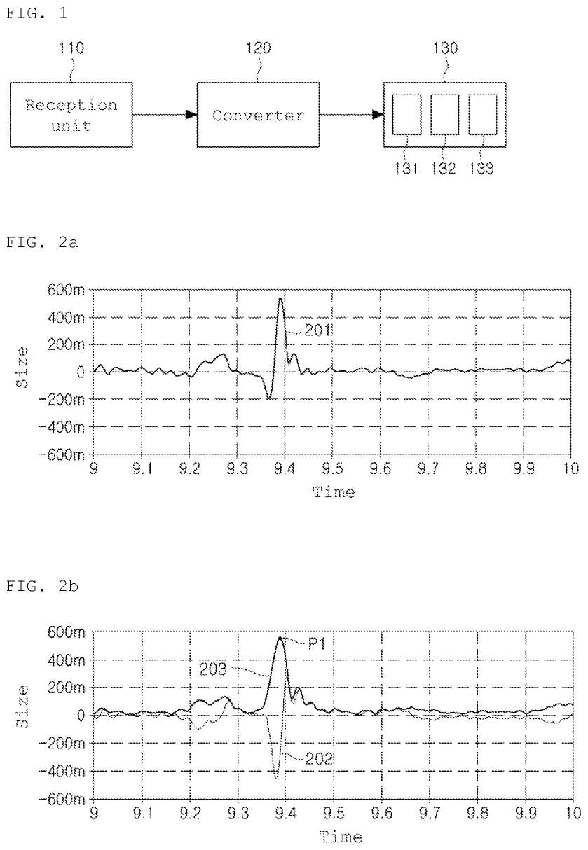

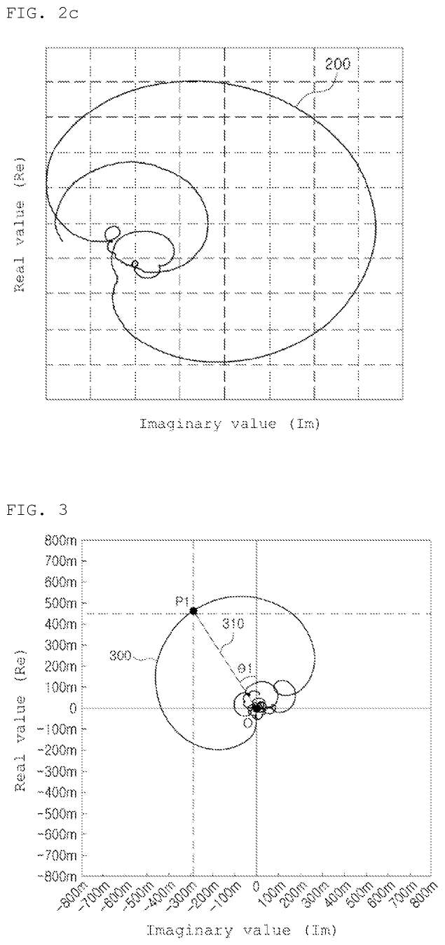

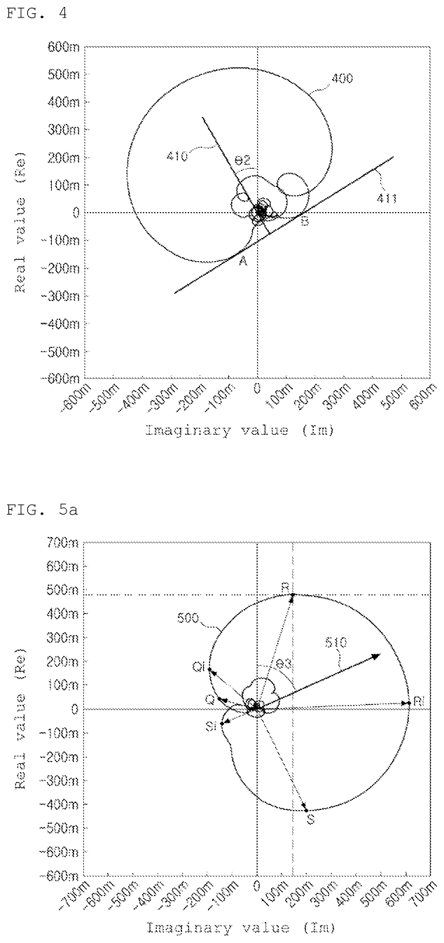

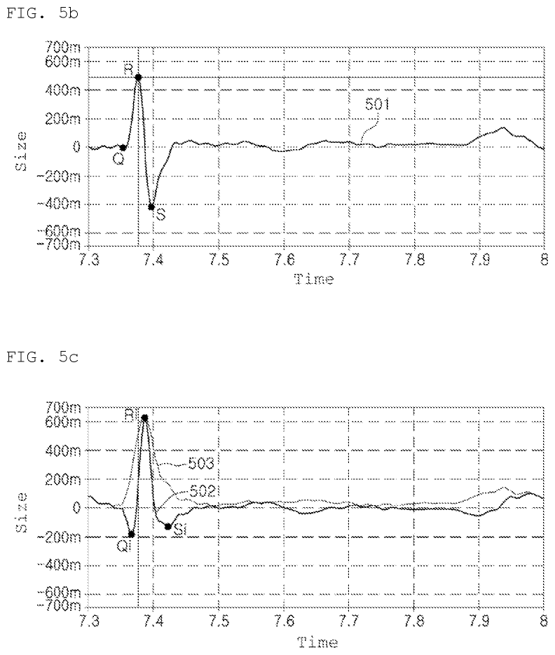

FIG. 1 is a block diagram of an ECG axial deviation measuring apparatus using Hilbert transform according to an embodiment of the present invention; FIG. 2a is a diagram illustrating an exemplary ECG signal according to an embodiment of the present invention; FIG. 2b is a diagram illustrating an exemplary Hilbert-transformed ECG signal and an envelope according to an embodiment of the present invention; and FIG. 2c is a diagram illustrating an exemplary Nyquist diagram according to an embodiment of the present invention.

First, as shown in FIG. 1, the ECG axial deviation measuring apparatus 100 using Hilbert transform according to an embodiment of the present invention may include: a reception unit 110 to receive the measured ECG signal; a converter 120 for Hilbert-transforming the received ECG signal; and a control unit 130 to calculate an electrocardiography (ECG) axial deviation based on the Hilbert-transformed ECG signal.

Specifically, the reception unit 110 may receive the ECG signal measured from the outside and transmit the received ECG signal to the converter 120. The ECG signal is, for example, as shown by reference numeral 201 in FIG. 2a.

Meanwhile, the converter 120 may execute Hilbert transform for the ECG signal received from the reception unit 110.

The above-described Hilbert transform may shift only a phase by +π/2 at the negative frequency and −π/2 at the positive frequency, while maintaining an amplitude of the ECG signal. Real signals can be extended to complex dimensions, thus making it easier to analyze the magnitude and phase. That is, assuming that any real signal is x(t) and the Hilbert-transformed signal is x\hat{ }(t), the signal extended in the complex dimension xₚ(t)=x(t)+jx\hat{ }(t) can be obtained. The Hilbert-transformed ECG signal is, for example, as indicated by reference numeral 202 of FIG. 2b.

Next, the control unit 230 may obtain an electrocardiography (ECG) axial deviation based on the Hilbert-transformed ECG signal. To this end, the control unit 130 may include a Nyquist diagram creation module 131, an envelope creation module 132 and a control module 133.

First, the Nyquist diagram creation module 131 of the control unit 130 may create a Nyquist diagram as a time response curve in which the ECG signal value is a real value and the Hilbert-transformed ECG signal value is an imaginary value. The created Nyquist diagram may be transmitted to the control module 133. In general, it should be noted that the Nyquist diagram means a frequency response curve to frequency whereas, in the present invention, it is a time response curve to time.

FIG. 2c shows a Nyquist diagram 200 prepared with the ECG signal as a real value (Y-axis) and Hilbert-transformed ECG signal as an imaginary value (X-axis). In FIG. 2c, real values are indicated on the Y-axis and imaginary values are indicated on the X-axis. However, it is apparent to those skilled in the art that real values may be indicated on the X-axis and while representing imaginary values on the Y-axis.

The envelope creation module 132 of the control unit 130 may create envelopes for the ECG signal 201 and the Hilbert-transformed ECG signal 202. The created envelope may be transmitted to the control module 133.

FIG. 2b shows an envelope 203 for the ECG signal and the Hilbert-transformed ECG signal. The envelope 203 is a root mean square (RMS) value of the ECG signal 201 and the Hilbert-transformed ECG signal 202, and may be a rooted value of the sum of the squares of the ECG signal 201 and the Hilbert-transformed ECG signal 202, respectively.

Finally, the control module 133 of the control unit 230 may calculate ECG axial deviation based on at least one of the Nyquist curve and the envelope.

According to an embodiment of the present invention, the control module 133 may obtain the angle of a straight line that connects a point corresponding to the peak value of the envelope in the Nyquist diagram with the origin of the Nyquist diagram, as the ECG axial deviation.

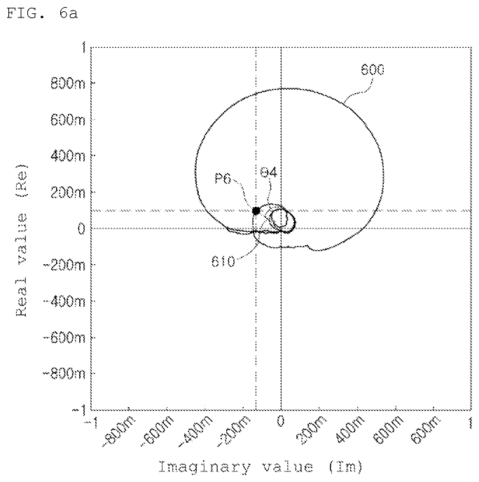

FIG. 3 illustrates a process of calculating the ECG axial deviation according to an embodiment of the present invention.

As shown in FIG. 3, the control module 133 may calculate the angle θ1 of a straight line 310 that connects the point P1 corresponding to the peak value of the envelope (203 in FIG. 2b) of the Nyquist diagram 300 with the origin (o) of the Nyquist diagram 300, as the ECG axial deviation. For this purpose, Equation 1 below may be used.

ECG axial deviation=atn(Imag(P1′)/Real(P1′)) [Equation 1]

Herein, atn is an arc tangent value, Imag(P1′) is a value of the imaginary axis of P1′, and Real(P1′) is a value of the real axis of P1′.

Further, according to an embodiment of the present invention, the control module 133 may obtain the angle defined by a perpendicular line from the common tangent of two points of the Nyquist diagram to the origin of the Nyquist diagram, as the ECG axial deviation.

FIG. 4 illustrates a process of obtaining the ECG axial deviation according to an embodiment of the present invention.

As shown in FIG. 4, the control module 133 may calculate the angle θ2 defined by a perpendicular line 40 from the common tangent 411 of two points A and B of the Nyquist diagram 400 to the origin (o) of the Nyquist diagram 400, as the ECG axial deviation.

If there are two or more common tangents, the control module 133 may obtain the angle defined by a perpendicular line from the average tangent of the two or more common tangents to the origin of the Nyquist diagram, as the ECG axial deviation.

On the other hand, according to an embodiment of the present invention, the control module 133 may calculate the angle of a vector, which is obtained by adding the sum of vectors up to each point corresponding to each peak value of Q wave, R wave and S wave of ECG signal in the Nyquist diagram, and the sum of vectors up to each point corresponding to each peak value of Qi wave, Ri wave and Si wave of Hilbert-transformed ECG signal in the Nyquist diagram, as the ECG axial deviation.

FIG. 5a to 5c illustrate a process of obtaining ECG axial deviation according to an embodiment of the present invention, wherein FIG. 5a is a Nyquist diagram 500, FIG. 5b shows ECG signal 501 and FIG. 5c shows Hilbert transformed ECG signal 502 and envelope 503.

As shown in FIGS. 5a to 5c, the control module 133 may calculate the angle θ3 of a vector 510, which is obtained by adding the sum of vectors up to each point corresponding to each peak value of Q wave, R wave and S wave of the ECG signal in the Nyquist diagram 500, and the sum of vectors up to each point corresponding to each peak value of Qi wave, Ri wave and Si wave of the Hilbert-transformed ECG signal in the Nyquist diagram, as the ECG axial deviation.

The above embodiments describe a method of obtaining the ECG axial deviation by targeting the QRS wave. However, the ECG axial deviation can also be obtained from T and P waves as well, and each axial deviation is classified according to the target wave. Therefore, when targeted the QRS wave, it may be referred to as QRS wave axial deviation. Further, when targeted the T wave and P wave, these may be referred to as T-wave axial deviation and P-wave axial deviation, respectively.

The T-wave axial deviation and the P-wave axial deviation may also be obtained through at least one of the methods described above. Hereinafter, the process of obtaining the T-wave axial deviation and the P-wave axial deviation through the first method will be described.

According to an embodiment of the present invention, the control module 133 may calculate the angle of a straight line that connects a point corresponding to the peak value of T wave of the envelope in the Nyquist diagram with the origin of the Nyquist diagram, as the ECG T wave axial deviation.

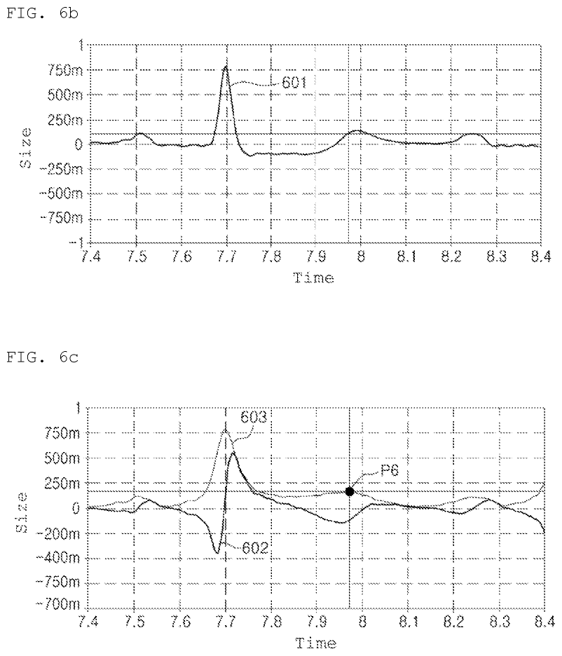

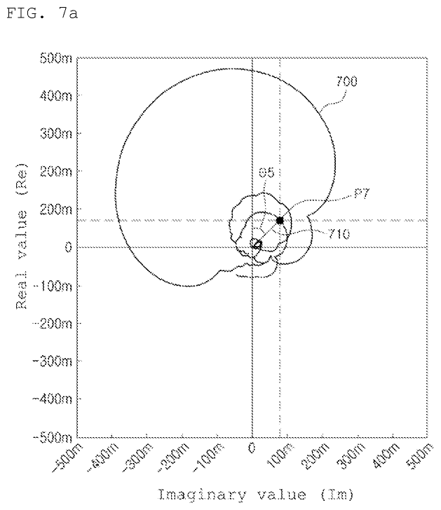

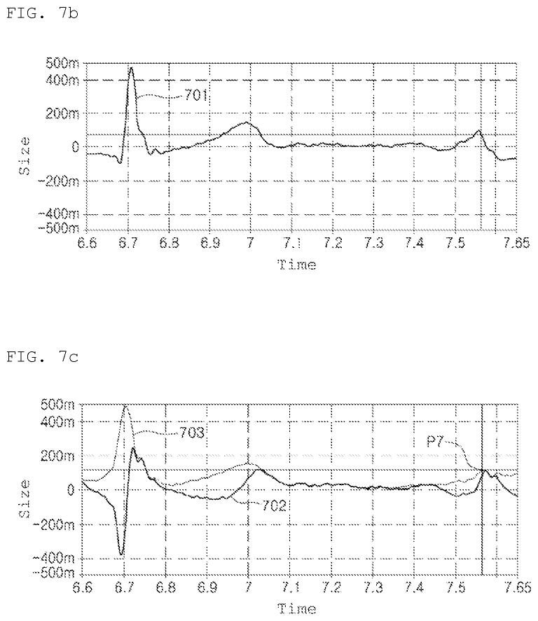

FIGS. 6a to 6c illustrate a process of obtaining ECG axial deviation according to an embodiment of the present invention, wherein FIG. 6a is a Nyquist diagram 600, FIG. 6b shows ECG signal 601 and FIG. 6c shows Hilbert-transformed ECG signal 602 and the envelope 603.

As shown in FIGS. 6a to 6c, the control module 133 may determine the angle θ4 of a straight line 610 that connects the point P6 corresponding to the peak value of T wave of the envelope in the Nyquist diagram 600 with the origin (o) of the Nyquist diagram, as the ECG T wave axial deviation.

Similarly, according to an embodiment of the present invention, the control module 133 may determine the angle of a straight line that connects a point corresponding to the peak value of P wave of the envelope in the Nyquist diagram with the origin of the Nyquist diagram, as the ECG P wave axial deviation.

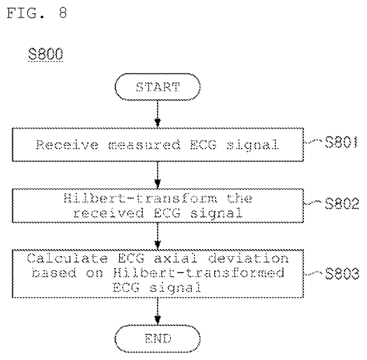

FIGS. 7a to 7c illustrate a process of obtaining an ECG axial deviation according to an embodiment of the present invention, wherein FIG. 7a is a Nyquist diagram 700, FIG. 7b shows ECG signal 701 and FIG. 7c shows Hilbert-transformed ECG signal 702 and the envelope 703.

As shown in FIGS. 7a to 7c, the control module 133 may determine the angle 65 of a straight line 710 that connects the point P7 corresponding to the peak value of P wave of the envelope in the Nyquist diagram 700 and the origin (o) of the Nyquist diagram, as the ECG P wave axial deviation.

As described above, according to an embodiment of the present invention, the received ECG signal is Hilbert-transformed, and ECG (electrocardiography) axial deviation is obtained based on the Hilbert-transformed ECG signal so that ECG axial deviation can be calculated only using a single lead without using multiple leads of extremity induction.

Meanwhile, FIG. 8 is a flowchart illustrating a method for measuring ECG axial deviation using Hilbert transform according to an embodiment of the present invention.

Hereinafter, a method for measuring ECG axial deviation using Hilbert transform according to an embodiment of the present invention will be described in detail with reference to FIGS. 1 to 8. However, for the sake of simplification of the invention, a description of the content overlapping with those previously described in FIGS. 1 to 7 will be omitted.

The ECG axial deviation measuring method using Hilbert transform according to an embodiment of the present invention may be started by receiving the ECG signal measured by the reception unit 110 (S801). The received ECG signal may be transmitted to the converter 120.

Next, the converter 120 may execute Hilbert transform for the received ECG signal (S802). The Hilbert-transformed ECG signal may be transmitted to the control unit 130.

Finally, the control unit 130 may obtain the ECG axial deviation based on the Hilbert-transformed ECG signal (S803).

According to an embodiment of the present invention, the control unit 130 may obtain the angle of a straight line that connects a point corresponding to the peak value of the envelope in the Nyquist diagram with the origin of the Nyquist diagram, as the ECG axial deviation.

Further, according to an embodiment of the present invention, the control unit 130 may obtain the angle defined by a perpendicular line from the common tangent of two points of the Nyquist diagram to the origin of the Nyquist diagram, as the ECG axial deviation.

Further, according to an embodiment of the present invention, the control unit 130 may determine the angle of a vector, which is obtained by adding the sum of vectors up to each point corresponding to each peak value of Q wave, R wave and S wave of the ECG signal in the Nyquist diagram, and the sum of vectors to each point corresponding to each peak value of Qi wave, Ri wave and Si wave of the Hilbert-transformed ECG signal in the Nyquist diagram, as the ECG axial deviation.

Further, according to an embodiment of the present invention, the control unit 130 may calculate the angle of a straight line that connects a point corresponding to the peak value of T wave of the envelope in the Nyquist diagram with the origin of the Nyquist diagram, as the ECG T wave axial deviation.

Further, according to an embodiment of the present invention, the control unit 130 may calculate the angle of a straight line that connects a point corresponding to the peak value of P wave of the envelope in the Nyquist diagram with the origin of the Nyquist diagram, as the ECG P wave axial deviation.

As described above, according to an embodiment of the present invention, the received ECG signal is Hilbert-transformed, and ECG axial deviation is obtained based on the Hilbert-transformed ECG signal so that ECG axial deviation can be calculated using only a single lead without using multiple leads of extremity induction.

In the description of the present invention, “˜part” to “˜module” may be implemented by various ways, for example, a processor, program instructions executed by the processor, software module, microcode, computer program product, logic circuit, application-specific integrated circuit, firmware, or the like.

The ECG axial deviation measurement method using Hilbert transform according to the embodiment of the present invention described above may be produced as a program to be executed by a computer and then may be stored in a computer-readable recording medium. Examples of the computer-readable recording medium include ROM, RAM, CD-ROM, magnetic tape, floppy disk, optical data storage device, or the like. Further, the computer-readable recording medium is distributed in a computer system connected through a network, so that the computer-readable code can be stored and executed in a distributed manner. Further, a functional program, code, and code segments for implementing the above method can be easily inferred by programmers in the art to which the present invention pertains.

The present invention is not limited by the above-described embodiments and the accompanying drawings. It is intended to limit the scope of rights to be protected by the appended claims, and it will be apparent or obvious to those skilled in the art that various forms of substitution, modification and change can be made without departing from the technical spirit of the present invention described in the claims.

1. An apparatus for measuring electrocardiography (ECG) axial deviation using Hilbert transform, comprising:
a reception unit to receive the measured ECG signal;
a converter for Hilbert-transforming the received ECG signal; and
a control unit to calculate an electrocardiography (ECG) axial deviation based on the Hilbert-transformed ECG signal.

2. The apparatus according to claim 1, wherein the control unit includes:
a Nyquist diagram creation module that creates a Nyquist diagram using a value of the ECG signal as a real value and a Hilbert-transformed value of the ECG signal as an imaginary value; and
a control module for calculating the ECG axial deviation based on the created Nyquist diagram.

3. The apparatus according to claim 2, wherein the control unit further includes:
an envelope creating module that creates an envelope for the ECG signal and the Hilbert-transformed ECG signal.

4. The apparatus according to claim 3, wherein the control module calculates an angle of a straight line that connects a point corresponding to a peak value of the envelope in the Nyquist diagram with an origin of the Nyquist diagram, as the ECG axial deviation.

5. The apparatus according to claim 2, wherein the control unit calculates an angle defined by a perpendicular line from a common tangent of two points of the Nyquist diagram to an origin of the Nyquist diagram, as the ECG axial deviation.

6. The apparatus according to claim 5, wherein, when two or more common tangents are present, the control unit calculates an angle defined by a perpendicular line from an average tangent of the two or more common tangents to the origin of the Nyquist diagram, as the ECG axial deviation.

7. The apparatus according to claim 2, wherein the control unit calculates an angle of a vector, which is obtained by adding the sum of vectors up to each point corresponding to each peak value of Q wave, R wave and S wave of the ECG signal in the Nyquist diagram, and the sum of vectors up to each point corresponding to each peak value of Qi wave, Ri wave and Si wave of the Hilbert-transformed ECG signal in the Nyquist diagram, as the ECG axial deviation.

8. The apparatus according to claim 3, wherein the control unit calculates the angle of a straight line that connects a point corresponding to the peak value of T wave of the envelope in Nyquist diagram with the origin of the Nyquist diagram, as the ECG T wave axial deviation.

9. The apparatus according to claim 3, wherein the control unit calculates the angle of a straight line that connects a point corresponding to the peak value of P wave of the envelope in the Nyquist diagram with the origin of the Nyquist diagram, as the ECG P wave axial deviation.

10. A method for measuring ECG axial deviation using Hilbert transform, comprising:
a first step of receiving the measured ECG signal in a reception unit;
a second step of Hilbert-transforming the received ECG signal in a converter; and
a third step of obtaining an ECG axial deviation based on the Hilbert-transformed ECG signal in a control unit.

11. A computer-readable recording medium in which a program for executing the method as set forth in claim 10.
